(19) United States
(12) Patent Application Publication
(10) Pub. No.: US 2022/0273397 A1
(43) Pub. Date: Sep. 1, 2022

(54) ELECTROSURGICAL HANDHELD DEVICE, AND CONTACT BODY FOR AN ELECTROSURGICAL HANDHELD DEVICE

(71) Applicant: OLYMPUS WINTER & IBE GMBH, Hamburg (DE)

(72) Inventors: Christian BROCKMANN, Hollenstedt (DE); Christoph KNOPF, Stockelsdorf (DE)

(73) Assignee: OLYMPUS WINTER & IBE GMBH, Hamburg (DE)

(21) Appl. No.: 17/682,669

(22) Filed: Feb. 28, 2022

(30) Foreign Application Priority Data
Feb. 26, 2021 (DE) 10 2021 104 612.2

(51) Int. Cl. A61B 90/70; A61B 18/14

(52) U.S. Cl. CPC A61B 90/70; A61B 18/149; A61B 2018/00178

(57) ABSTRACT

An electrosurgical handheld devices, and contact bodies of electrosurgical handheld devices, prove very difficult to clean. Cleaning them is very laborious, particularly when they have bores and receptacles of small diameters. Even mechanical cleaning cannot guarantee that the necessary degree of cleaning is achieved. The invention makes available an electrosurgical handheld device and a contact body that can be cleaned particularly easily and thoroughly and in a time-efficient manner. This is achieved by the fact that the contact body for an electrosurgical handheld device has at least one receptacle that is designed as a continuous bore through the contact body.

The invention relates to a contact body for electrosurgical handheld devices according to the preamble of claim 1. The invention also relates to an electrosurgical handheld device according to claim 11.

Electrosurgical handheld devices, for example resectoscopes, are used mainly for endoscopic applications in urology or gynecology, there preferably for treatment in the region of the bladder, the uterus or the prostate. However, the field of use of these instruments is not limited to these regions of the human body and instead also includes the treatment of further organs in the lower part of the human abdomen.

The instruments of the kind described here, for example resectoscopes, have a working element as standard. To treat the diseased tissue, the resectoscope is inserted with an elongate shaft through an opening into the body of the patient. Various medical instruments for treating and/or examining the patient can be arranged in this shaft tube. For example, an electrode to which radiofrequency alternating current can be applied and which is positioned at a distal end of an electrode carrier, can be guided through the shaft for radiofrequency surgery. For the manipulation or cutting of tissue, the electrode carrier with the electrode is movable relative to the shaft tube and along a shaft axis, such that, by moving the electrode on or through the tissue, the latter is manipulated.

The electrode carrier is furthermore coupled with its proximal end to the working element. This handling of the working element permits the cutting movement of the electrode. The working element has a movably mounted contact body, which is also designated as a slide. On this contact body, the electrode carrier can be mechanically and releasably coupled to an electrical contact. By way of this mechanical connection, the electrode carrier or the electrode can also be supplied with electrical energy. For this purpose, the contact body has a bore or opening shaped like a blind hole, into which an electrical contact of the electrode carrier can be guided for the releasable connection. This opening or this blind hole is designed in such a way that the RF voltage can be applied via an adjoining plug connector. For this purpose, a plug is usually fitted into the socket and is in turn connectable to an RF generator via a line or a cable.

The working element and also the electrode are actuated or longitudinally displaced by an operator. For this purpose, the working element is assigned a grip unit with a first gripping means and a second gripping means. To actuate the working element, the operator grasps the first gripping means, and also the second gripping means that can have a finger unit or a thumb ring. The first gripping means can be fastened to a stationary main body of the working element. The second gripping means can be fastened to the contact body.

The movement of the working element takes place counter to a spring tension of a spring, which in the working element of the type in question is designed usually as a leaf spring or as a leg spring. One end of this spring is fastened to the contact body or the slide, and the other end is fastened to an end body or an optical guide plate of a strengthening tube. The nature of the springs or the type of actuation of this spring mechanism depends on whether the working element is an active or passive working element. Whereas the spring in the case of an active working element is designed as a compression spring, in the case of a passive working element it is designed as a tension spring.

The cutting by the electrode is usually effected by a pulling-back movement of the working element. In the case of the active working element, the electrode is for this purpose pulled back (in the proximal direction) against the spring force of the spring. By contrast, in the case of the passive working element, the electrode is first of all pushed forward (in the distal direction) counter to the spring force, in order then to cut through the tissue during the return movement (in the proximal direction) caused by the relaxation of the spring.

An optical unit can also be guided through the shaft of the instruments described here. The rod-like or shaft-like optical unit is guided from the proximal end through a strengthening tube of the working element into the shaft. Embodiments are known in which the optical unit is guided as a rod lens system or as an optical fiber through the shaft from a proximal end to the distal end. The distal end of the optical unit is directed directly to the surgical region or the site of action of the electrode. At the proximal end of the optical unit, the operator can observe the treatment through an eyepiece or a camera.

The at least one electrical contact of the electrode system is then connected to the contact body and then pushed through or into the shaft. By actuation of the second gripping means relative to the first gripping means, the contact body together with the electrode carrier can thus be moved to and fro along the shaft axis over a strengthening tube.

At the proximal end of the strengthening tube, the latter is welded to an optical guide plate. The contact body is arranged movably on the strengthening tube. For this purpose, the contact body has a corresponding bore for the strengthening tube, parallel to the shaft axis. This bore is dimensioned such that the contact body or the slide can be pushed easily over the strengthening tube.

The cleaning of the contact receptacle or blind hole for the contact of the electrode carrier has proven to be a particular disadvantage. The cleaning is made very difficult in particular because of the small hole diameter and also because of the use of electrical tulip contacts inside the blind hole. Even with mechanical cleaning, it is not possible to guarantee that the necessary degree of cleaning is achieved. In particular, residues of liquids or particles that have entered the receptacle during the treatment or also during the cleaning process can be removed only with difficulty, if indeed at all. Besides the problem of inadequate conditioning or cleaning, particles and liquid remaining in the receptacle can also lead to contact problems between the electrode carrier and the contact in the receptacle.

Proceeding from this, the object of the invention is to make available an electrosurgical handheld device and a contact body that can be cleaned in a particularly simple, thorough and time-efficient manner.

A contact body for achieving this object has the features of claim 1. Accordingly, provision is made that the contact body for an electrosurgical handheld device has at least one receptacle, which is designed as a continuous bore through the contact body. This continuous receptacle extending from an end face of the contact body to an opposite end face of the contact body can be cleaned particularly thoroughly and easily. This channel-like bore extending through the entire contact body can be flushed especially thoroughly with a cleaning medium, specifically in such a way that neither particles nor residual liquid remain in the receptacle. Such a receptacle chosen for the electrical contact can also be dried particularly efficiently, such that, after the cleaning process, the interior of the receptacle is free of residues.

Preferably, provision is moreover made that the contact body has two receptacles which are each designed as continuous bores through the entire contact body. A contact body of such configuration is designed to receive or electrically contact an electrode carrier having two electrical contacts. These electrical contacts, which each run to the distally arranged electrode, in particular through electrode casing tubes, can be an active contact and a return contact or the active electrode and the return electrode.

Preferably, provision is made that the two receptacles for the two aforementioned electrical contacts are oriented parallel to each other and also parallel to a longitudinal axis of the handheld device or electrode carrier and lie in a common plane. By virtue of this symmetry, it is not just the structure of the handheld device and of the electrode carrier that is particularly compact and space-saving but also the shape of the contact body. In particular, through the parallel arrangement of the receptacles to a shaft axis and therefore optionally also to a bore for receiving a strengthening tube for the optical unit, the coupling and uncoupling of the electrode carrier to/from the contact body is particularly advantageous. Thus, by means of this relative arrangement, it is not only possible to achieve contacting that is reliable but also contacting that is easy to operate. Even by applying just a slight force, the two contacts can be inserted into the receptacles and also removed again. Equally, the mechanical coupling of the contacts in the contact body is sufficiently strong even to withstand forces that occur parallel or transverse to the longitudinal axis during the treatment.

In particular, the invention provides that at least one receptacle has, on an inner wall, an electrical contact which is connectable to a generator via a plug connector, in particular via a plug socket, and an electrical connection, the plug connector preferably being integrated in the contact body. The plug connector can be arranged in the contact body in a manner perpendicular or parallel to the receptacle. Preferably, the plug connector is guided from underneath through the contact body to the channel-like receptacle. In addition, it can be particularly advantageous optionally to contact both receptacles by corresponding external plugs. Through this contacting of the two receptacles, it is possible to generate a particularly defined and stable electrical potential at the ends of the electrode carrier. It is equally conceivable that the two plugs or plug connections are guided from the proximal end face into the at least one receptacle of the contact body. Alternatively, it is also conceivable that a cable on which a plug connector is arranged is routed out from the contact body.

Moreover, provision can be made according to the invention that the electrical contact is designed, inside the at least one receptacle, as a cylindrical, hollow cylindrical or annular electrical contact, preferably as a lamella contact or helical spring contact. With these types of contacts, the electrical contacting of the contacts of the electrode carrier can be produced in a particularly reliable manner. In addition, it is also conceivable that the receptacles are assigned another form of contact for the electrical contacting. In a particularly advantageous illustrative embodiment of the invention, provision can moreover be made that a distance of the at least one contact from an end face of the contact body, in particular from an outlet of the at least one receptacle, measures at least 2 mm, preferably at least 4 mm, in particular at least 6 mm.

In a further preferred illustrative embodiment of the invention, provision can be made that a mechanical latching means, for releasably coupling at least a proximal end of the electrode carrier, extends into at least one receptacle, the latching means being able to be actuated, preferably locked and/or released, via a maneuvering means, in particular a spring-pretensioned button. Preferably, only one of the two contacts of the electrode carrier is locked with the latching means, so as to avoid an electrical short circuit between the two contacts. It is conceivable that the corresponding proximal region on the electrode carrier, where the latching is performed, has a cross-sectional deformation, for example a notch or a recess, in which the latching means can engage with a latching action. This latching connection is configured in such a way that it can be carried out with one hand by the operator.

In addition, in an advantageous development, provision can be made that the bore of the at least one receptacle has a funnel-like shape at a distal outlet, in particular that an end face of the contact body has a funnel-like depression around the bore of the at least one receptacle. This funnel-like shape of the inlet region of the receptacle makes it easier to insert the contacts of the electrode carrier into the receptacles. When joining the electrode carrier to the contact body, it is sufficient that the two proximal ends of the electrode carrier are guided into the funnel. The oblique shape of the inner walls of the funnel then guides the contacts directly into the receptacles. This facilitates the contacting of the electrode carrier in particular during the operation.

Moreover, provision can be made according to the invention that a seal, in particular a fluid seal, is arranged in the receptacles or bores, in front of or behind the electrical contacts. By means of this seal, it is possible to avoid a situation where irrigation liquid flows out of the shaft through the receptacle of the contact body and possibly leads to an electrical short circuit during the treatment. By means of this seal, the liquid is at least for the most part kept out of the receptacle. Particularly preferably, this seal is arranged directly at the distal end of the receptacles.

It has been found that plastic, in particular PTFE, is particularly advantageous for the contact body on account of the material properties, such as low sliding resistance, high electrical resistance, smooth surface, and good processability. However, it is equally conceivable too that the contact body can be produced from another fluoropolymer, for example PFA. PEEK has also proven advantageous. These materials are also particularly suitable because of the fact that they can be cleaned particularly thoroughly.

The object mentioned at the outset is also achieved by the features of claim 11. Accordingly, provision is made that an electrosurgical handheld device, for example a resectoscope, with an electrode carrier which at a distal end has an electrode and at a proximal end has at least one electrical contact, has a contact body as claimed in at least one of claims 1-10, in which the at least one proximal end of the electrode carrier can be contacted.

Preferably, provision is made that at least one component, in particular a stop, of the handheld device, which is arranged in the proximal or distal direction to the contact body or strikes the contact body, is made of an electrically non-conductive material or has an electrically non-conductive layer, preferably a coating. This can prevent irrigation liquid from passing through the channel-like receptacle during the treatment of the patient and generating an electrical connection between the contact of the electrode carrier and, for example, the returning electrical potential. Alternatively, it is possible to lengthen the receptacle, e.g. if the proximal end of the receptacle does not open out at the proximal end face but instead, for example, on the underside.

It is moreover conceivable that an electrically conductive component, in particular a stop, of the handheld device is separated by an air gap from the contact body, preferably from an outlet of at least one channel-like receptacle of the contact body, the air gap having a width of at least 1 mm, in particular at least 2 mm. For example, it is conceivable that a stop surface is spaced radially apart from the outlet of the at least one channel-like receptacle of the contact body.

In addition, provision is preferably made that a distance between the electrode and a distal end of a shaft, in particular the optical unit or an electrode casing tube, is less than a distance between a proximal end of the electrode carrier and a proximal end face of the contact body and/or less than a distance between an electrical contact and at least one end face of the contact body. This is intended to ensure that, during the treatment, a plasma ignites between the electrode and the neutral electrode and not in the contact body between the proximal end of the electrode carrier and the proximal end face of the contact body.

A preferred illustrative embodiment of the invention is explained in more detail below with reference to the drawing, in which:

FIG. 1 shows a schematic view of a resectoscope,

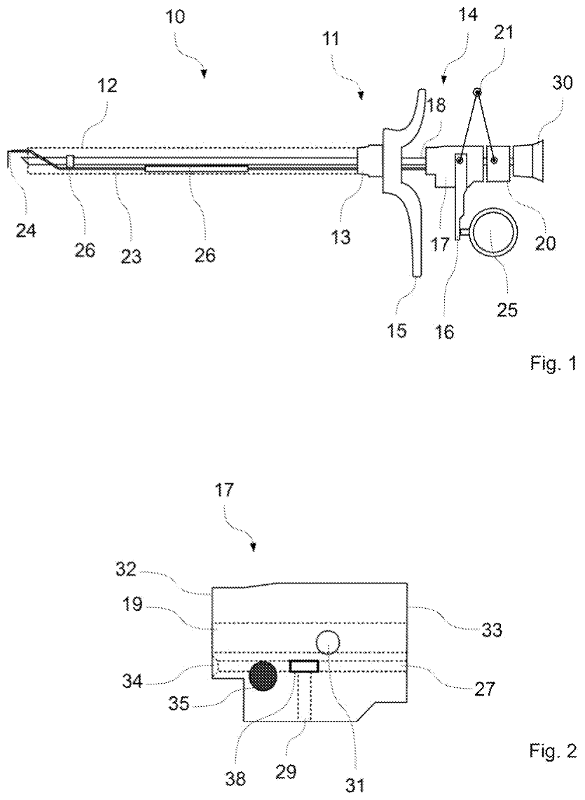

FIG. 2 shows a side view of a contact body,

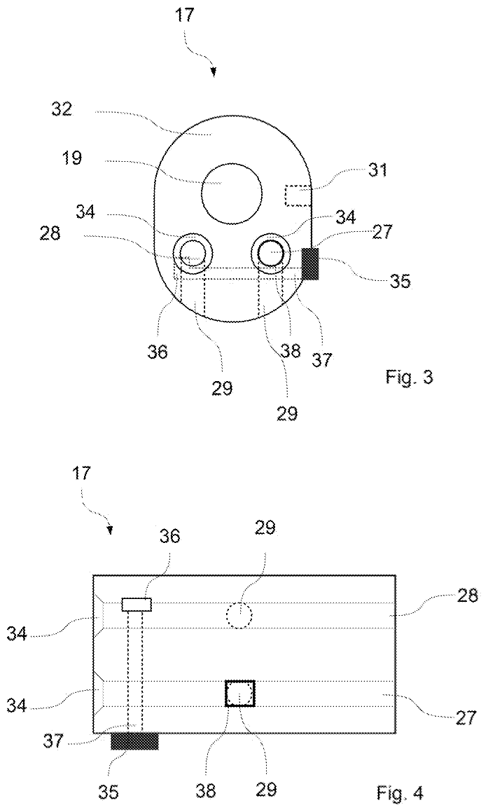

FIG. 3 shows a view of an end face of the contact body according to FIG. 2, and

FIG. 4 shows a section through the contact body according to FIG. 2.

A possible illustrative embodiment of an electrosurgical handheld device, namely a resectoscope 10, is depicted highly schematically in FIG. 1. The resectoscope 10 has a working element 11 on which an elongate, tubular shaft 12 can be fastened. This shaft 12 is indicated by hatching in FIG. 1 and is fastened with a proximal end to a main body 13 of the working element 11.

The working element 11 has, in addition to the main body 13, a grip unit 14. This grip unit 14 has a first gripping means 15 and a second gripping means 16. While the first gripping means 15 is arranged fixedly on the main body 13, the second gripping means 16 is assigned to a contact body 17 in the illustrative embodiment of the working element 11 shown here. It is conceivable that the second gripping means 16 is screwed firmly on the contact body 17. For this purpose, the contact body 17 has a corresponding bore 31 in a wall.

The contact body 17 is guided slidingly on a tubular optical guide 18. For this purpose, the contact body 17 has a bore 19 whose diameter is slightly greater than a diameter of the optical guide 18. Since the contact body 17 can move to and fro on the optical guide 18 along a longitudinal direction of the resectoscope 10 or a longitudinal axis of the shaft 12, the contact body 17 is also designated as a slide.

While the optical guide 18 is connected with a distal end to the main body 13 or an inner tube 22, an optical guide plate 20 is fastened at a proximal end of the optical guide 18. The tubular optical guide 18 extends through the optical guide plate 20, such that the optical guide 18 is accessible from the proximal direction.

The second gripping means 16 and the contact body 17 are connected to the optical guide plate 20 via a spring element 21. This spring element 21 can be a tension spring or a compression spring, depending on the configuration of the working element 11.

Starting from the main body 13, a tubular inner tube 22 extends in the distal direction. This inner tube 22 can also extend in the proximal direction through the main body 13 and be connected to the optical guide 18.

An electrode carrier 23 extends parallel to the inner tube 22. This electrode carrier 23 is guided through the main body 13 and with at least one proximal contact is mechanically and releasably coupled to the contact body 17. The electrode carrier 23 has an electrode 24 at a distal end. An electrical RF voltage can be applied to this electrode 24. The diseased tissue can be manipulated or cut by means of electrical energy arising at the electrode 24. For this purpose, the operator moves the second gripping means 16, having a thumb ring 25, relative to the first gripping means 15. For stabilizing the electrode carrier 23, the latter can be guided on the inner tube 22 by guides 26.

For applying the RF voltage to the electrode 24, a receptacle 27 of the proximal contact of the electrode carrier 23 can be electrically contacted. For this purpose, the contact body 17 has at least one plug connector 29, in particular a plug socket. This plug connector 29 is in electrical contact with at least one part of an inner wall of the receptacle 27. By way of a plug (not shown here), the contact body 17 can thus be connected by a cable to an RF generator.

For performing the intervention, a rod-like optical unit is guided through the inner tube 22 or optical guide 18. A distal end (not visible here) of this optical unit is directed in the direction of the electrode 24, so that the operator has a view of the manipulation of the tissue. This optical unit can be a rod lens system or an optical fiber. As is shown in FIG. 1, an eyepiece 30 or a camera is located at the proximal end of the optical unit.

FIG. 2 shows a possible illustrative embodiment of a contact body 17. This contact body 17 can be annular and also square. PTFE has proven to be a particularly preferred material for this contact body 17. However, other fluoropolymers with similar material properties are also conceivable. Among others reasons, PTFE is recommended because of the fact that it has a particularly low sliding friction. The contact body 17 has in fact a continuous bore 19 through which, in the assembled state of the working element 11, the optical guide 18 extends. When the working element 11 is actuated, the contact body 17 is guided along the optical guide 18. To ensure that this guiding carried out by the operator can be carried out without too much force having to be exerted, it is important to keep the sliding resistance low.

On an outer wall, the contact body 17 has a bore 31 into which the second gripping means 16 can be mounted. However, this bore 31 is not absolutely necessary. Instead, it is conceivable that the second gripping means 16 can also be connected in another way to the contact body 17.

According to the invention, a channel-like receptacle 27 extends through the contact body 17, specifically from one end face 32 to the other end face 33 of the contact body 17. The illustrative embodiment of the contact body 17 shown here has two of these channel-like receptacles 27, 28 (FIG. 3, FIG. 4). These two receptacles 27, 28 are oriented parallel to each other and also parallel to the bore 19. The receptacles 27, 28 are located in the same plane in the contact body 17 and are spaced apart from each other in such a way that the two proximal ends of the electrode carrier 23 can be guided into the receptacles 27, 28. These two proximal ends of the electrode carrier 23 each have an electrical contact, for example an active contact and a return contact.

In the receptacles 27, 28, these contacts of the electrode carrier 23 are not only mechanically fixed but also have a predefined electrical potential applied to them. For easier insertion of the contacts into the receptacles 27, 28, the ends of the receptacles 27, 28 each have a funnel-like depression or a funnel 34 at the end face 32. This funnel 34 greatly facilitates the coupling of the electrode carrier 23 to the contact body 17.

The inner walls of the receptacles 27, 28 have electrical contacts. These contacts can be lamella contacts or helical spring contacts, for example. So that electrical energy can be applied to these contacts inside the receptacles 27, 28, both receptacles 27, 28 are connected to a respective plug connector 29, in particular a two-pole coaxial plug or a corresponding socket for a coaxial plug. As is shown for example in FIGS. 2 and 3, these plug connectors 29 can be guided from underneath through the contact body 17 to the receptacles 27, 28. It is equally conceivable that the plug connector 29 extends parallel to the receptacles 27, 28 through the contact body 17. To start up the resectoscope 10, plugs connected to the generator (not shown) by an electrical conductor or by a cable have to be inserted into the plug connector 29.

In order not only to electrically contact the electrode carrier 23 in the contact body 17 but also to produce the mechanical locking to the contact body 17, a latching means 36 is assigned to at least one receptacle 28. This latching means 36 is connected to a button 35 via a shaft-like pin 37 (FIG. 4). The latching means 36 is spring-pretensioned and can be actuated by the button 35. By the actuation of the button 35, the latching means 36 is pressed out of the receptacle 28, such that the electrode carrier 23 is insertable with the two contacts into the contact body 17. The contact guided into the receptacle 28 has a recess into which the latching means 36 engages, as a result of which the mechanical coupling is produced. It is equally conceivable that, during the insertion of the electrode carrier 23 into the receptacle 28, the latching means 36 is pressed back and latches into the corresponding recess.

Preferably, the latching means 36 is assigned to only one receptacle 27, 28, in order to avoid the danger of an electrical short circuit between the two receptacles 27, 28.

The continuous channel-like receptacles 27, 28 are particularly easy to clean. Thus, an irrigation liquid can be flushed easily and effectively through the contact body 17 from one end face 32 to the other end face 33. The above-described disadvantage of particles in particular remaining in a blind hole, even after the cleaning procedure, can be completely eliminated here. Subsequent drying of the contact body 17 is also readily possible.

To prevent a leakage current or an electrical breakdown, components of the resectoscope 10 that are directed toward an end face 32, 33 of the contact body 17 can be made of an electrically insulating material or can have a corresponding coating. This prevents an electrical contact to a further electrical potential being produced, for example by irrigation liquid that could pass through the receptacles 27, 28 during the operation.

The contact body 17 is dimensioned in such a way that a distance between a contact part of the electrode, or the contact 38, and one of the end faces 32, 33 is greater than the distance between the electrode 24 and the nearest conductive component, in particular the optical unit or an electrode casing tube, to return potential. It can thus be ensured that the plasma ignites at the electrode 24 and not in the contact body 17.

1. A contact body for an electrosurgical handheld device, in particular a resectoscope, with at least one receptacle for a proximal end, having at least one electrical contact, of an electrode carrier of the handheld device, the contact being able to be mechanically and electrically coupled in the receptacle wherein the at least one receptacle is designed as a continuous bore through the contact body.

2. The contact body as claimed in claim 1, wherein two receptacles which are each designed as a continuous bore through the contact body.

3. The contact body as claimed in claim 2, wherein the two receptacles are oriented parallel to each other and also parallel to a longitudinal axis of the handheld device and lie in a common plane.

4. The contact body as claimed in claim 1, wherein at least one receptacle has, on an inner wall, an electrical contact which is connectable to a generator via a plug connector and an electrical connection, the plug connector being integrated in the contact body.

5. The contact body as claimed in claim 4, wherein the electrical contact is designed, inside the at least one receptacle, as a cylindrical, hollow cylindrical or annular electrical contact.

6. The contact body as claimed in claim 4, wherein a distance of the at least one contact from an end face of the contact body, from an outlet of the at least one receptacle, measures at least 2 mm.

7. The contact body as claimed in claim 1, wherein a mechanical latching means, for releasably coupling at least a proximal end of the electrode carrier, extends into at least one receptacle, the latching element being able to be actuated, via a maneuvering means.

8. The contact body as claimed in claim 1, wherein the bore of the at least one receptacle has a funnel-like shape at a distal outlet, in that an end face of the contact body has a funnel-like depression or a funnel around the bore of the at least one receptacle.

9. The contact body as claimed in claim 1, wherein a seal, is arranged in the bore, in front of or behind the electrical contact.

10. The contact body for an electrosurgical handheld device as claimed in claim 1, wherein the contact body is made of plastic.

11. An electrosurgical handheld device, a resectoscope, with an electrode carrier which at a distal end has an electrode and at a proximal end has at least one electrical contact, with a grip unit consisting of a first gripping means and a second gripping means, with a tubular shaft which is coupled with a proximal end to the first gripping means, with an optical guide for receiving an optical unit, and a contact body through which the optical guide can be guided, the second gripping means can be fastened, and in which the at least one electrical contact of the electrode carrier can be latched and/or electrically contacted, wherein a contact body as claimed in claim 1.

12. The electrosurgical handheld device as claimed in claim 11, wherein at least one component, a stop, of the handheld device, which is arranged in the proximal or distal direction to the contact body or strikes the contact body, is made of an electrically non-conductive material or has an electrically non-conductive layer.

13. The electrosurgical handheld device as claimed in claim 11, wherein an electrically conductive component, in particular a stop, of the handheld device is separated by an air gap from the contact body, the air gap having a width of at least 1 mm.

14. The electrosurgical handheld device as claimed in claim 11, wherein a distance between the electrode and a distal end of the shaft, the optical unit or an electrode casing tube, is less than a distance between a proximal end of the electrode carrier and a proximal end face of the contact body and/or less than a distance between an electrical contact and at least one end face of the contact body.
